(19) United States
(12) Patent Application Publication
(10) Pub. No.: US 2025/0218126 A1
(43) Pub. Date: Jul. 3, 2025

(54) MULTI-MODAL QUERY AND RESPONSE ARCHITECTURE FOR MEDICAL PROCEDURES

(71) Applicant: Intuitive Surgical Operations, Inc., Sunnyvale, CA (US)

(72) Inventors: Omid Mohareri, San Francisco, CA (US); Muhammad Abdullah Jamal, Sunnyvale, CA (US)

(73) Assignee: Intuitive Surgical Operations, Inc., Sunnyvale, CA (US)

(21) Appl. No.: 19/002,068

(22) Filed: Dec. 26, 2024

Related U.S. Application Data
(60) Provisional application No. 63/616,243, filed on Dec. 29, 2023.

(51) Int. Cl. G06T 19/00; G06F 40/40; G06V 10/82; G06V 20/40; G16H 50/70

(52) U.S. Cl. CPC G06T 19/00; G06V 10/82; G06V 20/41; G06V 20/46; G16H 50/70; G06F 40/40; G06T 2210/41; G06T 2210/56; G06T 2219/004; G06V 2201/03

(57) ABSTRACT

Aspects of this technical solution can receive a text prompt from a user, generate, based at least in part on a plurality of sets of data associated with at least one medical procedure, an output corresponding to the text prompt, where each of the plurality of sets of data has a different modality, where the plurality of sets of data comprises depth data, and provide the output for display.

### CROSS-REFERENCE TO RELATED APPLICATION

This application claims the benefit of, and priority to, U.S. Patent Application No. 63/616,243, filed Dec. 29, 2023, the full disclosure of which is incorporated herein in its entirety.

### TECHNICAL FIELD

The present implementations relate generally to medical devices, including but not limited to a multi-modal query and response architecture for medical procedures.

### INTRODUCTION

Surgeons, nurses, technicians, and staff of operating rooms must adhere to many medical protocols and constraints to ensure effective and responsive medical care of patients and to ensure the highest likelihood of desired and expected medical outcomes, including, but not limited to, completion of surgery or surgical procedures within the time allotted and in the absence of additional unexpected or unplanned medical interventions. However, given the complexity of the physical environment of the operating room and actions involved in surgery, conventional systems cannot provide a sufficient awareness of a procedure or effectively analyze past procedures to improve efficiency or outcomes of those procedures.

### SUMMARY

Systems, methods, apparatuses, and non-transitory computer-readable media are provided for generating responses to queries about aspects of a medical procedure (e.g., a surgical procedure) or a medical environment (e.g., an operating room (OR)). Multimodal data can include data having distinct formats (e.g., video, audio, text, medical procedure event data, medical procedure workflow data, medical environment workflow data, surgical workflow analytics data, medical procedure segmentation analytics data, 3D point cloud data, etc.) that can respectively be processed by various models configured to analyze those formats. For example, a model can correspond to a large language model that can analyze text prompt provided by a user. The text prompt can include, for example, “Was the room and equipment setup optimal for this procedure?” For example, a model can correspond to a neural network model that can analyze inputs such as videos of a given procedure performed in a given medical environment. The user could select or reference the video from a database of recordings or menu of available recordings, for example. Each of the models can generate respective features descriptive of the text prompt. Thus, a technical solution for a multi-modal query and response architecture for medical procedures is provided.

At least one aspect is directed to a system. The system can include one or more processors, coupled with memory. The system can receive a text prompt from a user. The system can generate, based at least in part on a plurality of sets of data associated with at least one medical procedure, an output corresponding to the text prompt, where each of the plurality of sets of data has a different modality, where the plurality of sets of data comprises depth data. The system can provide the output for display.

At least one aspect is directed to a system. The system can include one or more processors, coupled with memory. The system can receive multi-modal data that can include video data, analytics data, and metadata for one or more medical procedures each to update one or more models. The system can generate, using a first model configured to detect image features, a first feature that identifies an object in the video data for a plurality of medical procedures, where the video can include at least one of medical staff, a patient, a robotic system or instrument, or a medical environment (e.g., an operating room (OR)). The system can generate, using a second model, a second feature that identifies features in a text prompt. The system can generate, by a third model and based on the first feature and the second feature, an output responsive to the input text prompt, where the output comprises at least one of text or media content. The system can determine, based on the first feature and the second feature, a loss with respect to the output. The system can update at least one of the first model, the second model, and the third model based on the loss.

At least one aspect is directed to a non-transitory computer readable medium that can include one or more instructions stored thereon and executable by a processor. The processor can receive a text prompt from a user. The processor can generate, based at least in part on a plurality of sets of data associated with at least one medical procedure, an output corresponding to the text prompt, where each of the plurality of sets of data has a different modality, where the plurality of sets of data comprises depth data. The processor can provide the output for display.

At least one aspect is directed to a non-transitory computer readable medium that can include one or more instructions stored thereon and executable by a processor. The processor can receive multi-modal data can include video data, analytics data, and metadata for one or more medical procedures each to update one or more models. The processor can generate, using a first model configured to detect image features, a first feature that identifies an object in the video data for a plurality of medical procedures, where the video can include at least one of medical staff, a patient, a robotic system or instrument, or an operating room (OR). The processor can generate, using a second model, a second feature that identifies features in a text prompt. The processor can generate, by a third model and based on the first feature and the second feature, an output responsive to the input text prompt, where the output comprises at least one of text or media content. The processor can determine, based on the first feature and the second feature, a loss with respect to the output. The processor can update at least one of the first model, the second model, and the third model based on the loss.

At least one aspect is directed to a system. The system can include one or more processors, coupled with memory. The system can receive a text prompt from a user. The system can determine a text prompt feature for the text prompt. The system can identify, based at least in part on the text prompt feature, a plurality of sets of data associated with at least one medical procedure, where each of the plurality of sets of data has a different modality, where at least one set of the plurality of sets of data comprises depth data. The system can process the plurality of sets of data based on the text prompt feature. The system can generate, based on the processing of the plurality of sets of data, an output responsive to the text prompt feature.

At least one aspect is directed to a method. The method can include receiving a text prompt from a user. The method can include generating, based at least in part on a plurality of sets of data associated with at least one medical procedure, an output corresponding to the text prompt, where each of the plurality of sets of data has a different modality, where the plurality of sets of data comprises depth data. The method can include providing the output for display.

At least one aspect is directed to a method. The method can include receiving multi-modal data that can include video data, analytics data, and metadata for one or more medical procedures each to update one or more models. The method can include generating, using a first model configured to detect image features, a first feature that identifies an object in the video data for a plurality of medical procedures, where the video can include at least one of medical staff, a patient, a robotic system or instrument, or an operating room (OR). The method can include generating, using a second model, a second feature that identifies features in a text prompt. The method can include generating, by a third model and based on the first feature and the second feature, an output responsive to the input text prompt, where the output comprises at least one of text or media content. The method can include determining, based on the first feature and the second feature, a loss with respect to the output. The method can include updating at least one of the first model, the second model, and the third model based on the loss.

At least one aspect is directed to a method. The method can include receiving a text prompt from a user. The method can include determining a text prompt feature for the text prompt. The method can include identifying, based at least in part on the text prompt feature, a plurality of sets of data associated with at least one medical procedure, where each of the plurality of sets of data has a different modality, where at least one set of the plurality of sets of data comprises depth data. The method can include processing the plurality of sets of data based on the text prompt feature. The method can include generating, based on the processing of the plurality of sets of data, an output responsive to the text prompt feature.

### BRIEF DESCRIPTION OF THE FIGURES

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1 depicts an example system according to this disclosure.

FIG. 2 depicts an example first video query operation according to this disclosure.

FIG. 3 depicts an example second video query operation according to this disclosure.

FIG. 4A depicts an example first rendered query operation according to this disclosure.

FIG. 4B depicts an example second rendered query operation according to this disclosure.

FIG. 4C depicts an example third rendered query operation according to this disclosure.

FIG. 5 depicts an example temporal query operation according to this disclosure.

FIG. 6 depicts an example event-based query operation according to this disclosure.

FIG. 7 depicts an example event timeline data according to this disclosure.

FIG. 8 depicts an example data visualization query operation according to this disclosure.

FIG. 9 depicts an example data visualization output according to this disclosure.

FIG. 10 depicts an example cross-modal processor according to this disclosure.

FIG. 11 depicts an example method of multi-modal query and response for medical procedures according to this disclosure.

FIG. 12 depicts an example method of multi-modal query and response for medical procedures according to this disclosure.

FIG. 13 depicts an example method of multi-modal query and response for medical procedures according to this disclosure.

FIG. 14 depicts an example method of multi-modal query and response for medical procedures according to this disclosure.

### DETAILED DESCRIPTION

Aspects of this technical solution are described herein with reference to the figures, which are illustrative examples of this technical solution. The figures and examples below are not meant to limit the scope of this technical solution to the present implementations or to a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted to not obscure the present implementations. Terms in the specification and claims are to be ascribed no uncommon or special meaning unless explicitly set forth herein. Further, this technical solution and the present implementations encompass present and future known equivalents to the known components referred to herein by way of description, illustration, or example.

Aspects of this disclosure are directed to a system including one or more machine learning models that are combined, modified or connected to obtain a query including a plurality of inputs of a plurality of respective types. Aspects of this disclosure are directed to a system including one or more machine learning models that are combined, modified or connected to generate a response to the query, where the response includes a plurality of outputs of a plurality of respective types. For example, data of a plurality of types or differing types can be considered multi-modal data, as discussed herein. For example, a system can obtain a text query and a video of a medical procedure, and can generate a text response to the text query, and an annotated video that indicates aspects of the video relevant to the text query or the text response. For example, the neural network can generate features describing objects or motion in or across video frames. For example, the large language model can generate features (or “embeddings”) indicating semantic or grammatical elements in the text prompt. For example, the embeddings of the large language model can indicate that a user is querying about the layout of the medical environment. The models can provide their output features to a transformer model (e.g., a “cross-modal module”) that can determine characteristics of a response, and can instruct one or more output models to generate responses of various formats according to the characteristics. For example, the output models can receive output metrics that indicate content of responses according to particular formats. For example, the transformer model can generate layout metrics to a localization model to generate an image corresponding to a layout of a medical environment. The layout metrics can identify location and positions of given people and objects in a medical environment, according to the features of the neural network and the layout of those objects in video input. Thus, the transformer model can synthesize inputs having multiple types and having content directed to aspects of a medical environment, and can generate multiple types of output, based on the specific information elicited by a query from a user.

In some embodiments, the transformer model includes one or more fusion layers to combine features associated with different data formats into multimodal features. The transformer model can be configured to include one or more interfaces to receive features from one or more different types of models or data sources, and to combine the features received. For example, the transformer model can receive visual features from a neural network and point cloud embeddings from a data source of points clouds captured of the given medical environment during the given medical procedure. The transformer model can combine the features, for example, by concatenating features into an aggregate feature, to both deploy and train the model with integrated input from multiple types of input data processed by multiple types of models. Types of data can include text, video, images, point cloud data, 3D reconstructions of a medical environment based on the point cloud data, medical system (e.g., robotically-assisted medical system) data, analytics data, metadata associated with any of the types of data, or any combination thereof, but is not limited thereto. For example, depth-acquiring sensors may be strategically placed around the theater so that their resulting depth frames at each moment may be consolidated into a single three-dimensional virtual element model depicting objects in the medical environment. Examples of a three-dimensional virtual element model include a 3D reconstruction based on a point cloud (also referred to as three-dimensional point cloud data). Similarly, sensors may be strategically placed in the theater to focus upon regions of interest.

Examples of the analytics data include metrics calculated for workflow efficiency, metrics calculated for a number of medical staff members, metrics calculated for time of each phase or task, metrics calculated for motion, metrics calculated for room size and layout, timeline, and so on. Examples of the metadata include procedure type, hospital information, room information, medical staff information, medical staff experience, information for medical robotic systems and instruments, patient complexity, patient information (e.g., BMI, size, stage of sickness, organ information, and so on), and medical system data. For example, medical system data can include, but are not limited to, system events of medical systems, kinematic/motion of robotic systems. For example, system events can include, but are not limited to, timestamps of different activities, sequence of actions. The transformer model thus can include multiple interfaces to receive features generated by multiple types of artificial intelligence models, and can include multiple fusion layers to generate aggregated features according to multiple permutations that can be tailored to given configurations of outputs. For example, the transformer model can pair visual features with point cloud embeddings in a first fusion layer, and can pair instances of those same visual features with robot state data in a second fusion layer, to provide a technical improvement to increase accuracy of multimodal output with respect to the visual features. Accordingly, data streams can be segmented into a plurality of periods, including operative periods and nonoperative periods. Each nonoperative periods can include at least one phase. Each phase includes at least one task.

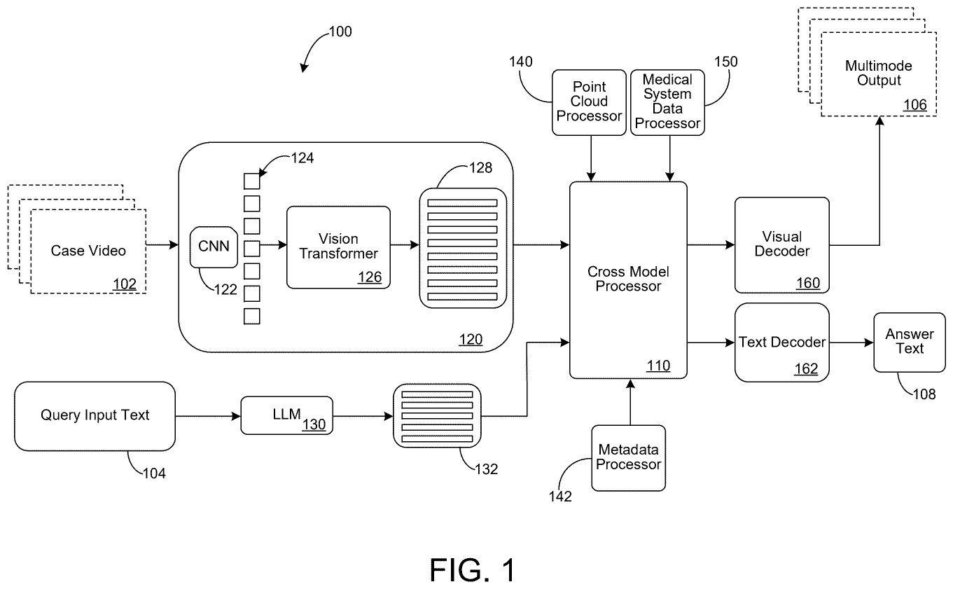

FIG. 1 depicts an example system according to this disclosure. As illustrated by way of example in FIG. 1, a system 100 can include at least a case video data 102, a query input text 104, a multimode output 106, an answer text 108, a cross-modal processor 110, a visual processing engine 120, a large language model (LLM) 130, a point cloud processor 140, a metadata processor 142, a medical system data processor 150, a visual decoder 160, and a text decoder 162.

The case video data 102 can include one or more images or one or more video segments including a depiction of one or more medical procedures. For example, the case video data 102 can include a video for a given medical procedure in a given medical environment (e.g., an OR). For example, the case video data 102 can include a video for a plurality of given medical procedures of a given type, in a given medical environment, or across a plurality of medical environments. The case video data 102 can include a depiction of one or more of medical environment, medical personnel, furniture, medical tools, robotically-assisted medical systems, manipulator assemblies and/or manipulator arms of robotically-assisted medical systems, or any combination thereof, but is not limited thereto. The query input text 104 can include a string or characters indicating a request to analyze an aspect of the case video data 102 or other data input to the system 100. For example, the query input text 104 can include a query directed to asking “how many” of any objects are associated with a given action, “how” a given action was performed, “where” a given object was located, “where” a given object was located with respect to an expected or required position of the object according to a medical procedure, “when” a given action was performed during a medical procedure, identify “what” property a given object has, “why” a given action was performed, or any combination thereof.

The multimode output 106 can include video data, a portion of video data, modified video data, annotated video data, an image object based on the case video data 102, any plurality thereof, or any combination thereof. For example, the multimode output 106 can include a portion of the case video data 102 that is responsive to a query. For example, the multimode output 106 can include an image depicting a layout of an operating room that corresponds to a layout the operating room depicted in the case video data 102 or the case video data 102 a given point in time. For example, a point in time can be a timestamp, a range of contiguous timestamps, or a plurality of timestamps. The answer text 108 can include a string or characters responsive to the query input text 104. For example, the answer text 108 can include response to a request to analyze an aspect of the case video data 102 or other data input to the system 100. For example, the answer text 108 can include a response to a query directed to asking “how many” of any objects are associated with a given action, “how” a given action was performed, “where” a given object was located, “where” a given object was located with respect to an expected or required position of the object according to a medical procedure, “when” a given action was performed during a medical procedure, identify “what” property a given object has, “why” a given action was performed, or any combination thereof.

The cross-modal processor 110 can fuse features output from one or more types of artificial intelligence models, and can generate one or more outputs having one or more types, which are responsive to a query. The cross-modal processor 110 can generate the outputs based on the fused features, to provide a technical improvement to artificial intelligence systems to achieve learning concurrently across multimodal input sand outputs, by a technical solution of at least providing a framework for machine learning processing by integrated features of a plurality of different data types into fused features. The cross-modal processor 110 can configurable fuse features based on content of input, type of input, weighting based on content, or weighting based on type or input, or any combination thereof. For example, the cross-modal processor 110 can determine a modality that is responsive to the text prompt, where the modality can include at least one of video data, an annotation of video data, an image, and a data visualization. For example, an annotation of video data can be bounding boxes around people in the case video data 102. For example, annotations can be indicative of visual features that provide spatial or temporal understanding of aspects of the video data. For example, a data visualization can be a chart or graph as discussed herein. For example, the cross-modal processor 110 can determine a modality that is responsive to the text prompt, where the modality can include at least one of video data, an annotation of video data, an image, and a data visualization. For example, an annotation of video data can be bounding boxes around people in the case video data 102. For example, a data visualization can be a chart or graph as discussed herein. For example, the cross-modal processor 110 can extract one or more features from one of more of the plurality of sets of data. The system can generate one or more fused features that each can include one or more of the features each having the different modality. The system can generate the output based on one or more of the fused features.

The visual processing engine 120 can generate one or more features indicative of one or more aspects of a visual input. For example, a visual input can include one or more images, or one or more frames of video, or any plurality or combination thereof. The visual processing engine 120 can include a convolutional neural network (CNN) 122, CNN output features 124, a vision transformer 126, and visual features 128. For example, the visual processing engine 120 can select, according to a determination that the plurality of sets of data can include video data, a neural network configured to extract features from the video data. The system can extract, by the neural network, the one or more features from the video data.

The convolutional neural network (CNN) 122 can include one or more connected neuron structures that can collectively identify one or more aspects of the visual input. For example, the CNN 122 can identify one or more people (e.g., medical personnel), medical devices or systems (e.g., robotically-assisted medical system), items of furniture, boundaries of a medical environment (e.g., an OR), and can collectively or individually be considered as content of the visual input. The CNN 122 can identify positions of the content of the visual input. For example, CNN 122 can identify that a surgeon is at a center of an OR. The CNN 122 can identify orientations of the content of the visual input. For example, the CNN 122 can identify that a surgeon is seated at a center of an OR facing a south end of the OR, or facing toward a console or input system of a robotically-assisted medical system in the OR. The CNN output features 124 can correspond to a first set of features that are output by the CNN 112 and are indicative of the aspects discussed above. The visual processing engine 120 is illustrated and described by way of example as including the CNN 122, but is not limited thereto. For example, the visual processing engine 120 can include a neural network or deep learning system other than a CNN.

The vision transformer 126 can include a machine learning model to generate one or more visual features based on input including one or more of the CNN output features 124. For example, the vision transformer 126 can receive the CNN output features 124 that are each descriptive of portions of the visual input, and can generate the visual features 128 that are correspondingly descriptive of portions of the visual input. The visual features 128 can correspond to a second set of features that are output by the vision transformer 126 and have a structure are compatible to be fused with output features of other artificial intelligence models. For example, the visual features 128 can be encoded with one or more alphanumeric descriptors that are also used to encode other features generated by other models. For example, the visual features 128 can be encoded with one or more alphanumeric descriptors that are also used to encode other features generated by other models.

The large language model (LLM) 130 can include a text transformer that provide text content responsive to a query. For example, the LLM 130 can include one or more processors to infer relationships between one or more characters, words, text fragments, or the like. The large language model (LLM) 130 can include text features 132. The text features 132 can correspond to a set of features that are output by the LLM 130 and have a structure are compatible to be fused with output features of other artificial intelligence models. For example, the text features 132 can be encoded with an encoding that at least partially matches the visual features 128. For example, the text features 132 can be encoded with one or more alphanumeric descriptors that are also used to encode other features generated by other models. For example, the text features 132 can be encoded with one or more alphanumeric descriptors that are also used to encode other features generated by other models.

The point cloud processor 140 can obtain data corresponding to point cloud data of an OR, and can generate a three-dimensional model of the data that depicts the OR. For example, the point cloud data corresponds to a 3D reconstruction of medical environment based on point cloud data/A 3D reconstruction can show a layout of a medical environment in three dimensions, at a given time before, during or after a medical procedure in the medical environment. The point cloud processor 140 can provide the 3D reconstructions in response to a specific text question (e.g., a prompt including an absolute or relative time, task, or phase of a medical procedure). For example, the point cloud processor 140 can include a plurality of points in a Cartesian coordinate system that each indicate that a surface exists at a position indicated by the respective coordinates of the points. The point cloud processor 140 can generate point cloud features that are each descriptive of portions of the point cloud data. The point cloud features can have a structure compatible to be fused with output features of other artificial intelligence models. For example, the point cloud features can be encoded with one or more alphanumeric descriptors that are also used to encode other features generated by other models. For example, the point cloud features can be encoded with an encoding that at least partially matches the visual features 128 and the text features 132.

The metadata processor 142 can obtain metadata corresponding to case video, query input text, point cloud data, robotic system data, or any combination thereof, and can generate or modify one or more features for the case video, query input text, point cloud data, or the robotic system data. The point cloud processor 140 can generate metadata features that are each descriptive of portions of the metadata. The point cloud processor 140 can modify features associated with given metadata to be indicative of the given metadata. For example, the point cloud processor 140 can modify a video feature to be indicative of metadata for that video that describes a medical procedure or medical environment depicted int eh video. The metadata features can have a structure compatible to be fused with output features of other artificial intelligence models. For example, the metadata features can be encoded with one or more alphanumeric descriptors that are also used to encode other features generated by other models. For example, the metadata features can be encoded with an encoding that at least partially matches the visual features 128 and the text features 132.

In some examples, case metadata can be stored in a memory device or a database. The memory device or the database can be provided for a scheduling or work allocation application that schedules hospital or OR processes and operations. For example, a user can input the case metadata, or the case metadata can be automatically generated using an automated scheduling application. The case metadata can be associated with other types of the information of a plurality of medical procedures such as the three-dimensional point cloud data, RGB image/video data, robot data, and so on. For example, other types of the information of a plurality of medical procedures captured for the same procedure time or scheduled time, in the same OR, with the same procedure name, with the same robot or instrument, or so on can be associated with the corresponding case metadata and can be processed together and displayed using the hierarchical structure together, in the same or different interfaces of a graphical user interface (GUI).

The medical system data processor 150 can obtain data associated with or generated by a medical system or device (e.g., a robotically-assisted medical system) or any component thereof, and can generate a model descriptive of the operation and/or state of the medical system over the course of a medical procedure. For example, the data associated with or generated by the medical system can include kinematics data (e.g., describing kinematics of robotic manipulator(s) of the medical system), data relating to medical instruments installed on or coupled to the medical system (e.g., information relating to instrument type, instrument status, etc.), image data generated by an imaging instrument (e.g., endoscopic imaging device) of the medical system. Such data can describe, for example, one or more of a position, orientation, displacement, velocity, acceleration, jerk, activation indication, or any combination thereof, of the medical system or any component thereof. For example, a component can include a manipulator assembly, a manipulator arm, an instrument supported by the manipulator assembly or manipulator arm, or the like, of the robotic system. Such data can further describe events on the medical system (e.g., medical system events data). Such events can include, for example, docking of the medical system to a patient, instrument exchanges, operator interactions with the medical system (e.g., surgeon head-in and head-out events on the input or surgeon console) The medical system data processor 150 can receive one or more robot features that are each descriptive of the operation and/or state of the robotic system. For example, the robot features can be indicative of at least one of a state of a robotic system, a state of a component of a robotic system (e.g., a manipulator or a seat), medical system operation features, or event features. For example, event features can be indicative of The robot features can have a structure compatible to be fused with output features of other artificial intelligence models. For example, the robot features can be encoded with one or more alphanumeric descriptors that are also used to encode other features generated by other models. For example, the robot features can be encoded with an encoding that at least partially matches the visual features 128, the text features 132, and the point cloud features.

The visual decoder 160 can generate one or more outputs including one or more images responsive to the query input text 104. For example, the visual decoder 160 can generate annotated video, a video segment, or a medical environment layout image as discussed herein. For example, the visual decoder 160 can include a localization engine that include one or more processors that converge toward an image output based on one or more fused features obtained from the cross-modal processor. Thus, the visual decoder 160 can generate the visual output based on features that are not limited to features indicative of video or images, to provide a technical improvement of generating responsive images or video based on input that is not limited to images or video.

The text decoder 162 can generate one or more outputs including text responsive to the query input text 104. For example, the text decoder 162 can generate the answer text 108 as discussed herein, based on one or more fused features obtained from the cross-modal processor. Thus, the text decoder 162 can generate the answer text 108 based on features that are not limited to features indicative of text, to provide a technical improvement of generating responsive text based on input that is not limited to text.

For example, the output of system 100 can include a text response and a visual annotation of video data, the text response can include a number responsive to the text prompt, and the plurality of sets of data comprises the video data. For example, the output can include a text response and a portion of video data within a time period, the text response and the time period each responsive to the text prompt, and the plurality of sets of data comprises the video data. For example, the output can include a text response and an image, and the text response can include at least a partial description of the image and is responsive to the text prompt. For example, the partial description of the image can include a description of at least one of an absolute location or a relative location within the medical environment of a first object in the plurality of sets of data, the relative location of the first object relative to a second object in the medical environment. For example, the description can be “the surgeon is standing at the south or right edge of the OR during prep.” For example, the partial description of the image can include a description of a layout of one or more objects in the plurality of sets of data, the image corresponds to a 3D reconstruction of a medical environment based on point cloud data, the medical environment corresponds to the medical procedure, and the plurality of set of data can include the point cloud data. For example, the point cloud processor 140 can provide the point cloud features to the cross-model processor 110, and the cross-modal processor 110 can generate a plan view layout of the OR that matches a 3D model of the point cloud data.

For example, the output can include a text response and a plurality of portions of video data, the text response can include a number responsive to the text prompt, a number of the plurality of portions of the video data corresponds to the number responsive to the text prompt, and the plurality of sets of data comprises the video data. For example, the portions of the video data can be segments of the case video data 102 during a pre-operative or pre-procedure period and a post-operative or post-procedure period. For example, the output can include a text response and a data visualization output, the text response can include at least a partial description of the data visualization output. For example, the data visualization output can include a chart or graph of time spent in pre-operative, intra-operative, and post-operative phases of a medical procedure, and the text response can be descriptive of the time spent in pre-operative, intra-operative, and post-operative phases of the medical procedure or surgery.

For example, the output can include a text response and a visual annotation of the video data, and the text response can include a number responsive to the text prompt. For example, the output can include a text response and a portion of the video data within a time period, the text response and the time period each responsive to the text prompt. For example, the output can include a text response and an image, the text response can include at least a partial description of the image and is responsive to the text prompt, and the media content comprises the image. For example, the partial description of the image can include a description of a location of an object in the multi-modal data. For example, the partial description of the image can include a description of a layout of one or more objects in the plurality of sets of data, and the image is based on the metadata. For example, metadata can include data obtained by the point cloud processor 140, data obtained by the medical system data processor 150, or any permutation thereof.

For example, the output can include a text response and a plurality of portions of the video data, the text response can include a number responsive to the text prompt, and a number of the plurality of portions of the video data corresponds to the number responsive to the text prompt. For example, the output can include a text response and a data visualization output, and the text response can include at least a partial description of the data visualization output.

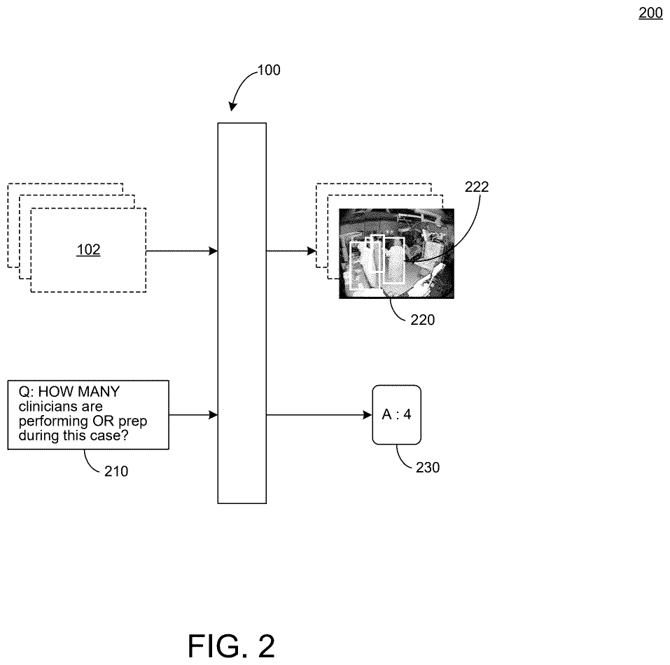

FIG. 2 depicts an example first video query operation according to this disclosure. As illustrated by way of example in FIG. 2, a first video query operation 200 can include at least a query input text 210, a video output 220, and an answer text output 230. The query input text 210 can be an example input according to the query input text 104. The query input text 210 includes a string of text according to the “how many” query, and asks, “How many clinicians are performing OR prep during this case?” The system 100 can receive the case video data 102 and the query input text 210. For example, the visual processing engine 120 receives the case video data 102 and the LLM 130 receives the query input text 210. In response to receiving the case video data 102 and the query input text 210, the system 100 can generate the video output 220, the visual annotations 222, and the answer text output 230. The video output 220 can be an example output according to the multimode output 106. For example, the video output 220 can be one or more frames of the case video data 102. The video output 220 can include visual annotations 222. The visual annotations 222 can include one or more modifications to the case video data 102 that result in the video output 220. For example, the video output 220 can be a portion of the case video data 102 at or between timestamps that are responsive to the query input text 210, and can include one or more bounding boxes or other visual information absent in the case video data 102. The answer text output 230 can be an example output according to the answer text 108. The answer text output 230 includes a string of text responsive to the “how many” query, and states, “4.”

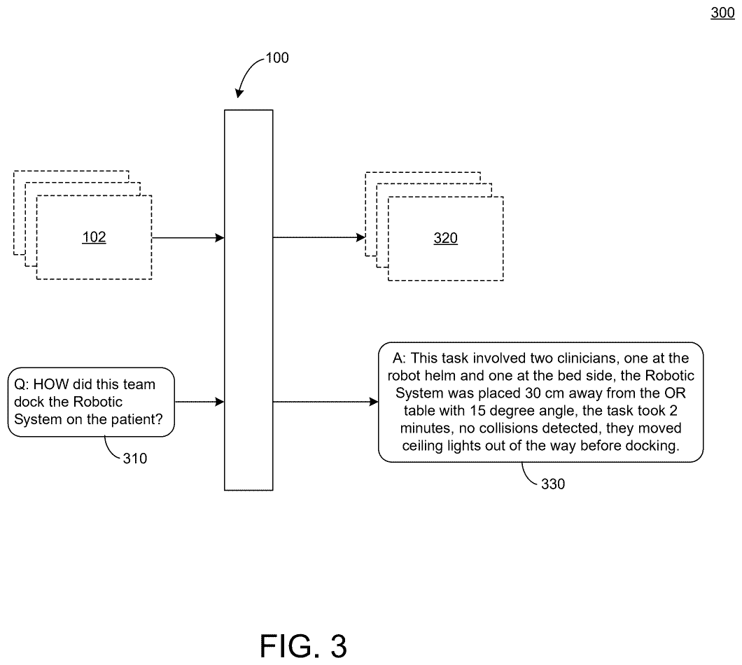

FIG. 3 depicts an example second video query operation according to this disclosure. As illustrated by way of example in FIG. 3, a second video query operation 300 can include at least a query input text 310, a video output 320, and an answer text output 330. The query input text 310 can be an example input according to the query input text 104. The query input text 310 includes a string of text according to the “how” query, and asks, “How did this team dock the robotic system on the patient?” The system 100 can receive the case video data 102 and the query input text 310. For example, the visual processing engine 120 receives the case video data 102 and the LLM 130 receives the query input text 310. In response to receiving the case video data 102 and the query input text 310, the system 100 can generate the video output 320 and the answer text output 330. The video output 330 can be an example output according to the multimode output 106. For example, the video output 330 can be one or more frames of the case video data 102. For example, the video output 330 can be a portion of the case video data 102 at or between timestamps that are responsive to the query input text 310. The answer text output 330 can be an example output according to the answer text 108. The answer text output 330 includes a string of text responsive to the “how” query, and states, “This task involved two clinicians, one at the robot helm and one at the bed side, the robot system was placed 30 cm away from the OR table with 15 degree angle, the task took 2 minutes, no collisions detected, they moved ceiling lights out of the way before docking.”

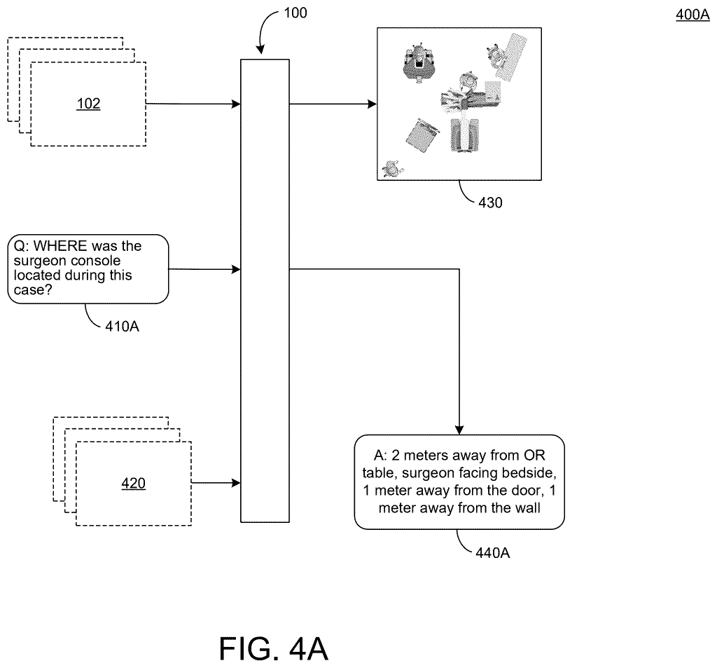

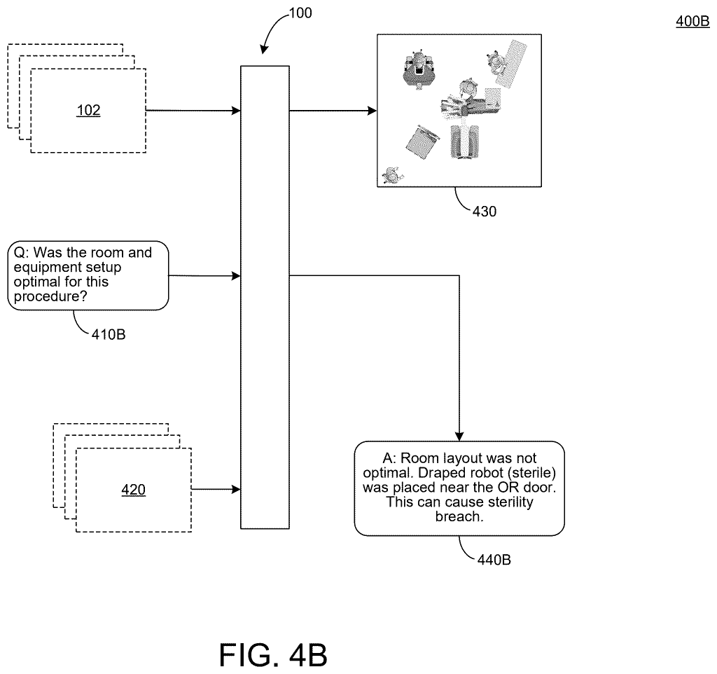

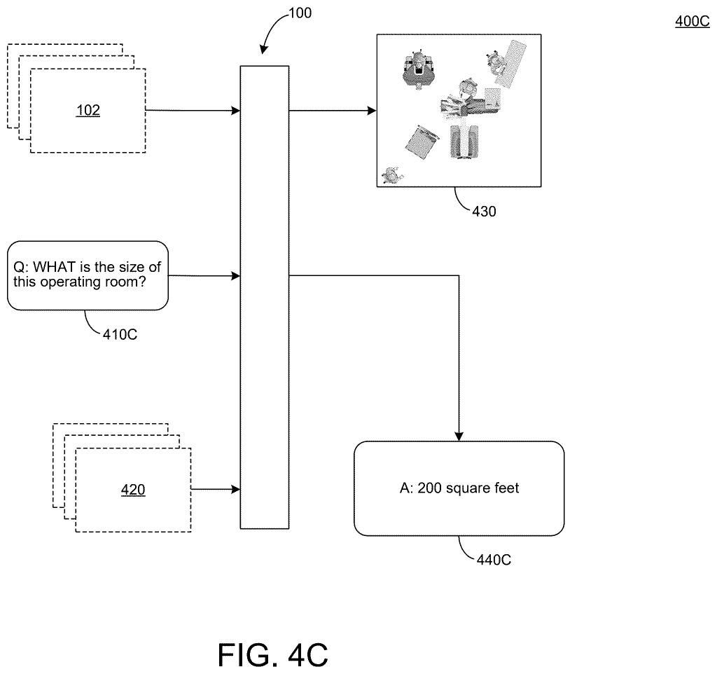

FIG. 4A depicts an example first rendered query operation according to this disclosure. As illustrated by way of example in FIG. 4A, a first rendered query operation 400A can include at least a query input text 410A, point cloud input frames 420, a rendered image output 430, and an answer text output 440A. The query input text 410A can be an example input according to the query input text 104. The query input text 410A includes a string of text according to the “where” query, and asks, “Where was the surgeon console located during this case?” The system 100 can receive the case video data 102, the query input text 410A, and the point cloud input frames 420. For example, the visual processing engine 120 receives the case video data 102, the LLM 130 receives the query input text 410A, and the point cloud processor 140 receives the point cloud input frames 420. In response to receiving the case video data 102, the query input text 410A, and the point cloud input frames 420, the system 100 can generate the rendered image output 430 and the answer text output 440A. The rendered image output 430 can be an example output according to the multimode output 106. For example, the rendered image output 430 can be one or more images based on the case video data 102. For example, the rendered image output 430 can depict one or more plan views of the OR, including one or more predetermined images or rendered images corresponding to specific people, furniture, tools, robots, or any combination thereof. The rendered image output 430 can include multiple images each corresponding to or between timestamps that are responsive to the query input text 410A. The answer text output 440A can be an example output according to the answer text 108. The answer text output 440A includes a string of text responsive to the “where” query, and states, “2 meters away from OR table, surgeon facing bedside, 1 meter away from the door, 1 meter away from the wall.”

FIG. 4B depicts an example second rendered query operation according to this disclosure. As illustrated by way of example in FIG. 4B, a second rendered query operation 400B can include at least a query input text 410B, and an answer text output 440B. The query input text 410B can be an example input according to the query input text 104. The query input text 410B includes a string of text according to the “where” query, and asks, “Was the room and equipment setup optimal for this procedure?” The system 100 can receive the case video data 102, the query input text 410B, and the point cloud input frames 420. For example, the visual processing engine 120 receives the case video data 102, the LLM 130 receives the query input text 410B, and the point cloud processor 140 receives the point cloud input frames 420. In response to receiving the case video data 102, the query input text 410B, and the point cloud input frames 420, the system 100 can generate the rendered image output 430 and the answer text output 410B. The answer text output 440B can be an example output according to the answer text 108. The answer text output 440B includes a string of text responsive to the “where” query, and states, “Room layout was not optimal. Draped robot (sterile) was placed near the OR door. This can cause sterility breach.”

FIG. 4C depicts an example third rendered query operation according to this disclosure. As illustrated by way of example in FIG. 4C, a third rendered query operation 400C can include at least a query input text 410C, and an answer text output 440C.

The query input text 410C can be an example input according to the query input text 104. The query input text 410C includes a string of text according to the “what” query, and asks, “What is the size of the operating room?” The system 100 can receive the case video data 102, the query input text 410C, and the point cloud input frames 420. For example, the visual processing engine 120 receives the case video data 102, the LLM 130 receives the query input text 410C, and the point cloud processor 140 receives the point cloud input frames 420. In response to receiving the case video data 102, the query input text 410C, and the point cloud input frames 420, the system 100 can generate the rendered image output 430 and the answer text output 410C. The answer text output 440C can be an example output according to the answer text 108. The answer text output 440C includes a string of text responsive to the “what” query, and states, “200 square feet.”

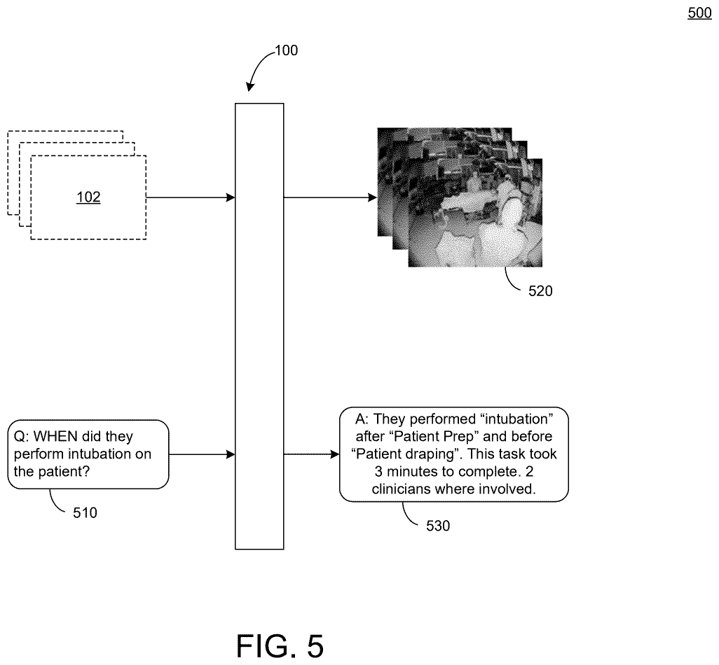

FIG. 5 depicts an example temporal query operation according to this disclosure. As illustrated by way of example in FIG. 5, a temporal query operation 500 can include at least a query input text 510, a video output 520, and an answer text output 530. The query input text 510 can be an example input according to the query input text 104. The query input text 510 includes a string of text according to the “when” query, and asks, “When did they perform intubation on the patient?” The system 100 can receive the case video data 102 and the query input text 510. For example, the visual processing engine 120 receives the case video data 102 and the LLM 130 receives the query input text 510. In response to receiving the case video data 102 and the query input text 510, the system 100 can generate the video output 520 and the answer text output 530. The video output 520 can be an example output according to the multimode output 106. For example, the video output 520 can be one or more frames of the case video data 102. For example, the video output 520 can be a portion of the case video data 102 at or between timestamps that are responsive to the query input text 510. The answer text output 530 can be an example output according to the answer text 108. The answer text output 530 includes a string of text responsive to the “when” query, and states, “They performed ‘intubation’ after ‘Patient Prep’ and before ‘Patient draping.’ This task took 3 minutes to complete. 2 clinicians were involved.”

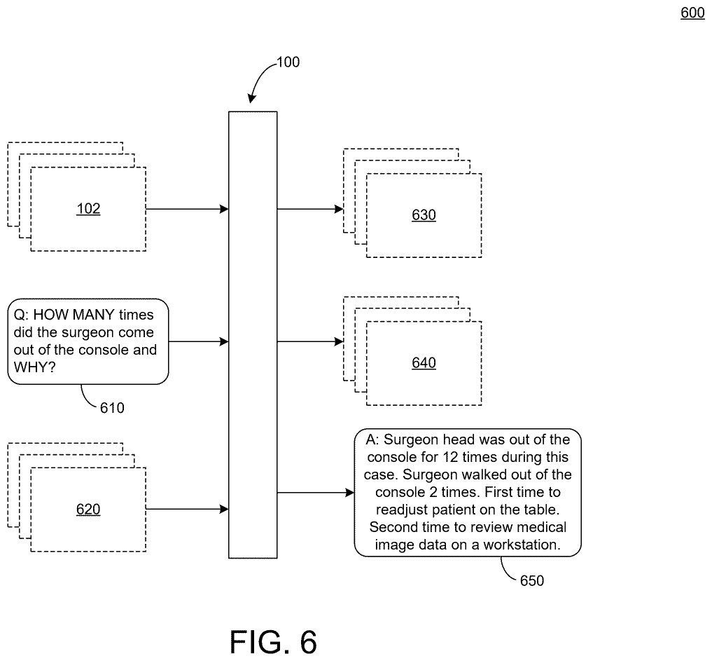

FIG. 6 depicts an example event-based query operation according to this disclosure. As illustrated by way of example in FIG. 6, an event-based query operation 600 can include at least a query input text 610, an event data input frames 620, a first video output 630, a second video output 640, and an answer text output 650. The query input text 610 can be an example input according to the query input text 104. The query input text 610 includes a string of text according to the “how many” query, and asks, “How many times did the surgeon come out of the console and why?” The system 100 can receive the case video data 102, the query input text 610, and the event data input frames 620. For example, the visual processing engine 120 receives the case video data 102, the LLM 130 receives the query input text 610, and the medical system data processor 150 receives the event data input frames 620. In response to receiving the case video data 102, the query input text 510, and the event data input frames 620, the system 100 can generate the first video output 630, the second video output 640, and the answer text output 650.

The first video output 630 and the second video output 640 can be an example output according to the multimode output 106. For example, the first video output 630 can be one or more frames of the case video data 102 between a first set of timestamps during which the surgeon came out of the console. For example, the second video output 640 can be one or more frames of the case video data 102 between a second set of timestamps during which the surgeon came out of the console. For example, the video output 650 can be a portion of the case video data 102 at or between timestamps that are responsive to the query input text 610. The answer text output 650 can be an example output according to the answer text 108. The answer text output 650 includes a string of text responsive to the “how many” query, and states, “Surgeon head was out of the console for 12 times during this case. Surgeon walked out of the console 2 times. First time to readjust patient on the table. Second time to review medical image data on a workstation.”

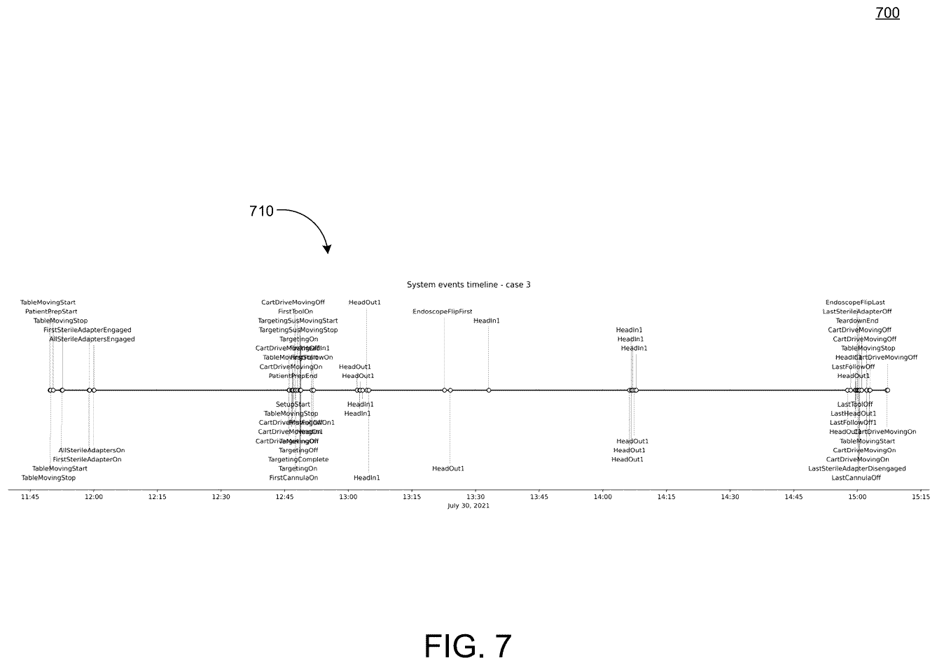

FIG. 7 depicts an example event timeline data according to this disclosure. As illustrated by way of example in FIG. 7, an event timeline data 700 can include at least a medical system (e.g., robotically-assisted medical system) event data 710. The robot event data 710 can include state information corresponding to change of state of various components of the robot. For example, the robot event data 710 can record states changes of the components of the robot as discussed herein, and can be associated with various timestamps that correspond to timestamps of the case video data 102 or the point cloud data. Thus, the robot event data 710 can be correlated by timestamp with the case video data 102 or the point cloud data.

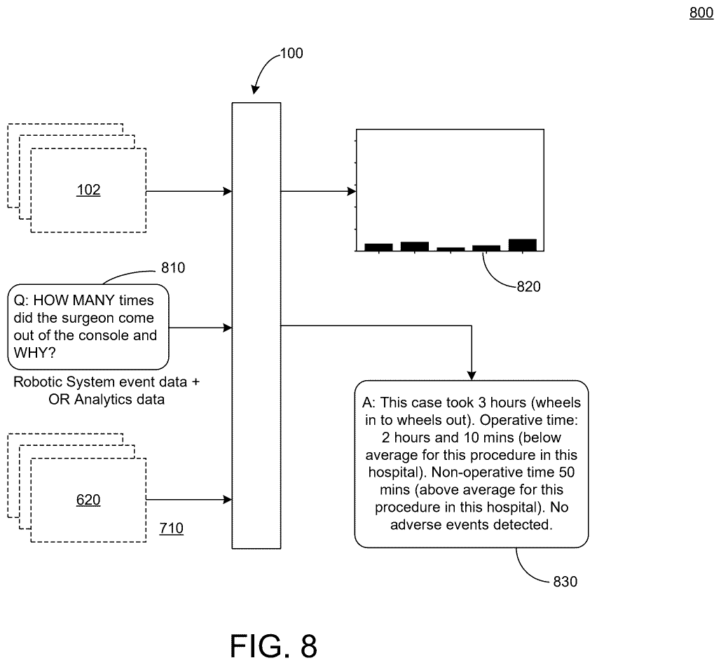

FIG. 8 depicts an example data visualization query operation according to this disclosure. As illustrated by way of example in FIG. 8, a data visualization query operation 800 can include at least a query input text 810, a data visualization output 820, and an answer text output 830. The query input text 810 can be an example input according to the query input text 104. The query input text 810 includes a string of text according to the “why” query, and asks, “Why did this case take so long?” The system 100 can receive the case video data 102, the query input text 810, and the event data input frames 620. For example, the visual processing engine 120 receives the case video data 102, the LLM 130 receives the query input text 810, and the medical system data processor 150 receives the event data input frames 620. In response to receiving the case video data 102, the query input text 810, and the event data input frames 620, the system 100 can generate the data visualization output 820 and the answer text output 830. The data visualization output 820 can be an example output according to the multimode output 106. For example, the data visualization output 820 can be a chart or graph based on the case video data 102. For example, the data visualization output 820 can divide the event data input frames 620 by phase of the medical procedure as discussed herein. The answer text output 830 can be an example output according to the answer text 108. The answer text output 830 includes a string of text responsive to the “how many” query, and states, “This case took 3 hours (wheels in to wheels out). Operative time: 2 hours and 10 mins (below average for this procedure in this hospital). Non-operative time 50 mins (above average for this procedure in this hospital). No adverse events detected.”

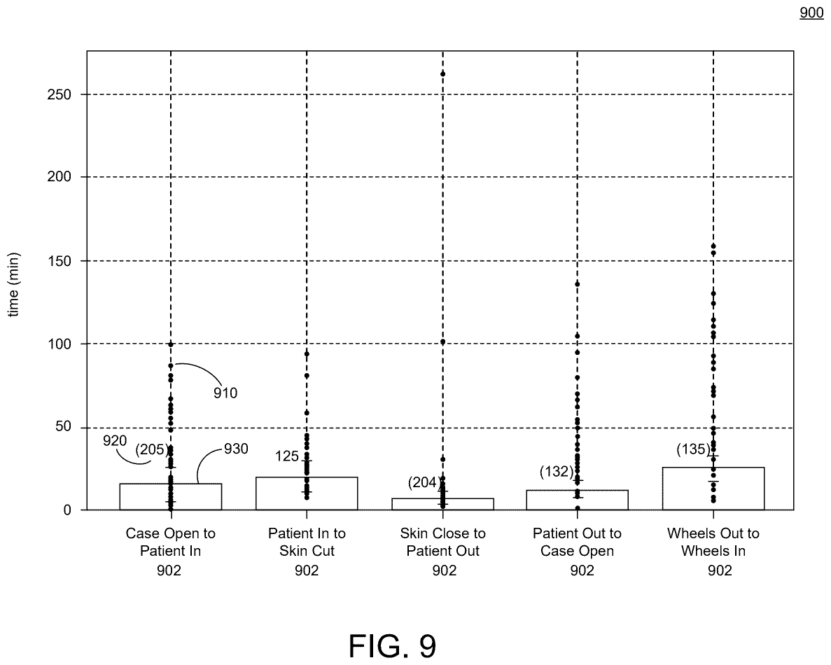

FIG. 9 depicts an example data visualization output according to this disclosure. As illustrated by way of example in FIG. 9, a data visualization output 900 can include at least an event phase data 902, a chart plot data 910, a chart annotation 920, and a chart bar data 930. The event phase data 902 can correspond to divisions of the event data input frames 620 according to phases of the medical procedure. For example, phases of the medical procedure can be correlated with timestamps of any data as discussed herein. The chart plot data 910 can identify the number of events per phase. The chart annotation 920 can identify a first parameter of the chart data, including an arithmetic mean, median, or mode of events in the phase. The chart bar data 930 can identify a second parameter of the chart data, including an arithmetic mean, median, or mode of events in the phase that is distinct from the first parameter. The data visualization output 900 is not limited to the data and annotation as discussed herein by way of example, and can include any data or metric presentable in a visualization as discussed herein.

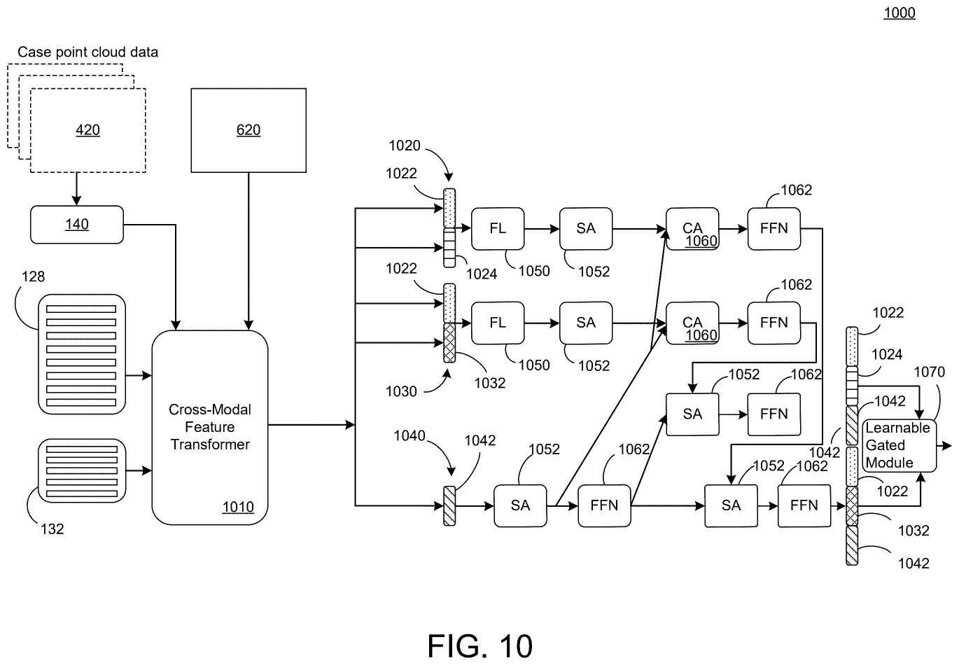

FIG. 10 depicts an example cross-modal processor according to this disclosure. As illustrated by way of example in FIG. 10, a cross-modal processor 1000 can include at least a cross-modal feature transformer 1010, a first fused feature 1020, a second fused feature 1030, an unfused text feature 1040, fusion layer (“FL”) processors 1050, self-attention (“SA”) processors 1052, cross-attention (“CA”) processors 1060, feed-forward networks (“FFNs”) 1062, and a learnable gated module 1070.

The cross-modal feature transformer 1010 can correspond at least partially in one or more of structure and operation to the cross-modal processor 110. The feature transformer can generate one or more fused features according to one or more feature fusion criteria. For example, a feature fusing criterion can correspond to a feature type to be fused with other feature types. Here, the cross-modal feature transformer 1010 fuse visual features with features of other types. However, this disclosure is not limited to the feature fusion combinations discussed herein. For example, the cross-modal feature transformer 1010 can generate the first fused feature 1020, the second fused feature 1030, and the unfused text feature 1040. The first fused feature 1020 can include a visual feature 1022, and a point cloud feature 1024. The visual feature 1022 can be a corresponding one of the visual features 128. The point cloud feature 1024 can be a corresponding one of the point cloud features. The second fused feature 1030 can include an event feature 1032. The event feature 1032 can be a corresponding one of the robot features. The unfused text feature 1040 can include a text feature 1042. The text feature 1042 can be a corresponding one of text feature 132. The cross-modal feature transformer 1010 can generate a plurality of fused and unfused features to allocate one or more, up to all, of the features received at the cross-modal feature transformer 1010, and is not limited to generating the features illustrated herein by way of example.

The cross-modal feature transformer 1010 can provide the features 1020, 1030, and 1040 to various components as discussed below. The cross-modal processor 110 can include the below features, but is not limited thereto. The cross-modal processor 110 is not limited to the configuration of processors as illustrated by way of example herein. The fusion layer processors 1050 can transform fused features for input to corresponding self-attention processors 1052. The cross-modal processor 110. The self-attention processors 1052 can execute a self-attention process on each of the corresponding fused features received from the corresponding fusion layer processors 1050, or the corresponding unfused features received from the cross-modal feature transformer 1010. The cross-attention processors 1060 can receive unfused or fused features from one or more self-attention processors 1052. The feed-forward networks 1062 can receive output from one or more of the self-attention processors 1052 or the cross-attention processors 1060. The feed-forward networks 1062 can be arranged in a cascading architecture with the self-attention processors 1052. The learnable gated module 1070 can receive the output of the last cascaded feed-forward network 1062. and can generate an output corresponding to one or more of the outputs as discussed herein, based on the fused features obtained via the cascaded network.

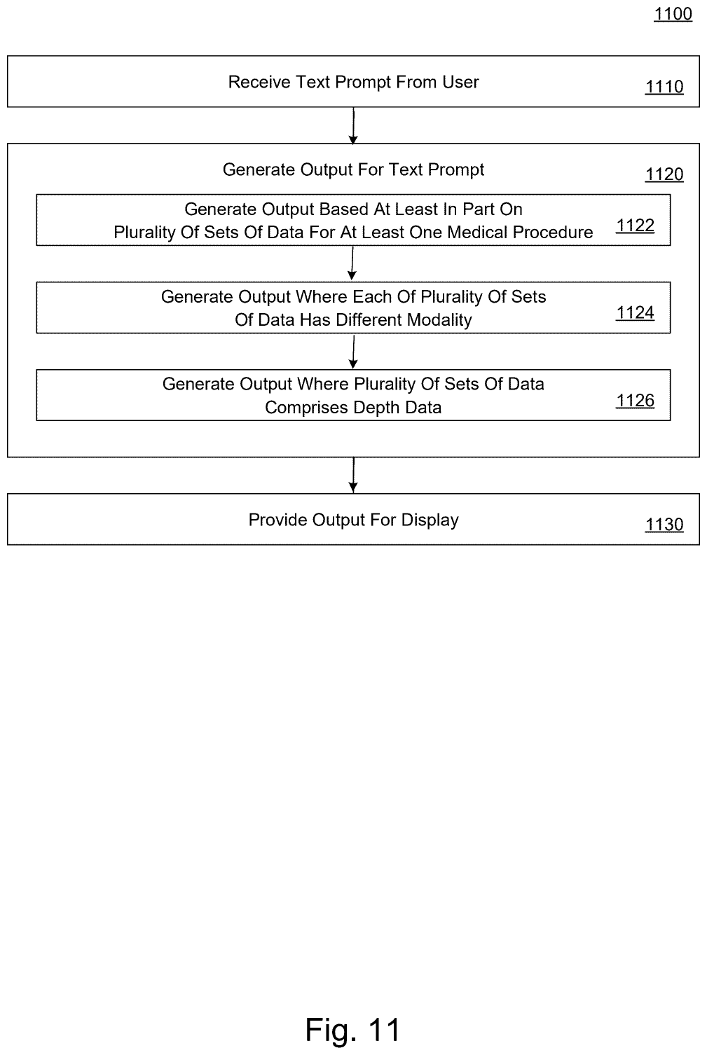

FIG. 11 depicts an example method of multi-modal query and response for medical procedures according to this disclosure. At least the system 100 can perform method 1100. At 1110, the system 100 can receive a text prompt from a user. At 1120, the system 100 can generate an output for the text prompt. At 1122, the system 100 can generate the output based at least in part on a plurality of sets of data for at least one medical procedure. At 1124, the system 100 can generate the output where each of the plurality of sets of data has a different modality. For example, a different modality can correspond to different data types or data formats. At 1126, the system 100 can generate the output where the plurality of sets of data comprises depth data. At 1130, the system 100 can provide the output for display.

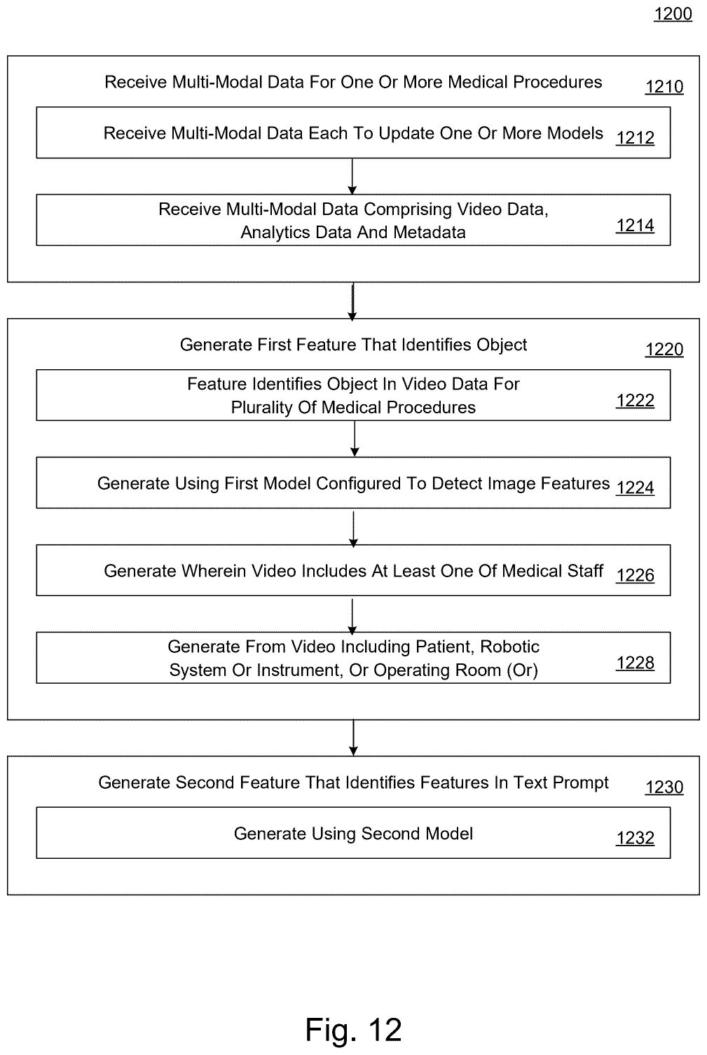

FIG. 12 depicts an example method of multi-modal query and response for medical procedures according to this disclosure. At least the system 100 can perform method 1200. At 1210, the system 100 can receive multi-modal data for one or more medical procedures. At 1212, the system 100 can receive multi-modal data each to update one or more models. At 1214, the system 100 can receive multi-modal data comprising video data, analytics data and metadata. At 1220, the system 100 can generate a first feature that identifies an object. At 1222, the system 100 can include a feature identifies an object in the video data for a plurality of medical procedures. At 1224, the system 100 can generate using a first model configured to detect image features. For example, the first model can correspond to the visual processing engine 120 or any component thereof. At 1226, generate wherein the video includes at least one of medical staff. At 1228, the system 100 can generate from video including a patient, a robotic system or instrument, or an operating room (or). At 1230, the system 100 can generate a second feature that identifies features in a text prompt. At 1232, the system 100 can generate the second feature using a second model. For example, the second model can correspond to the LLM 130.

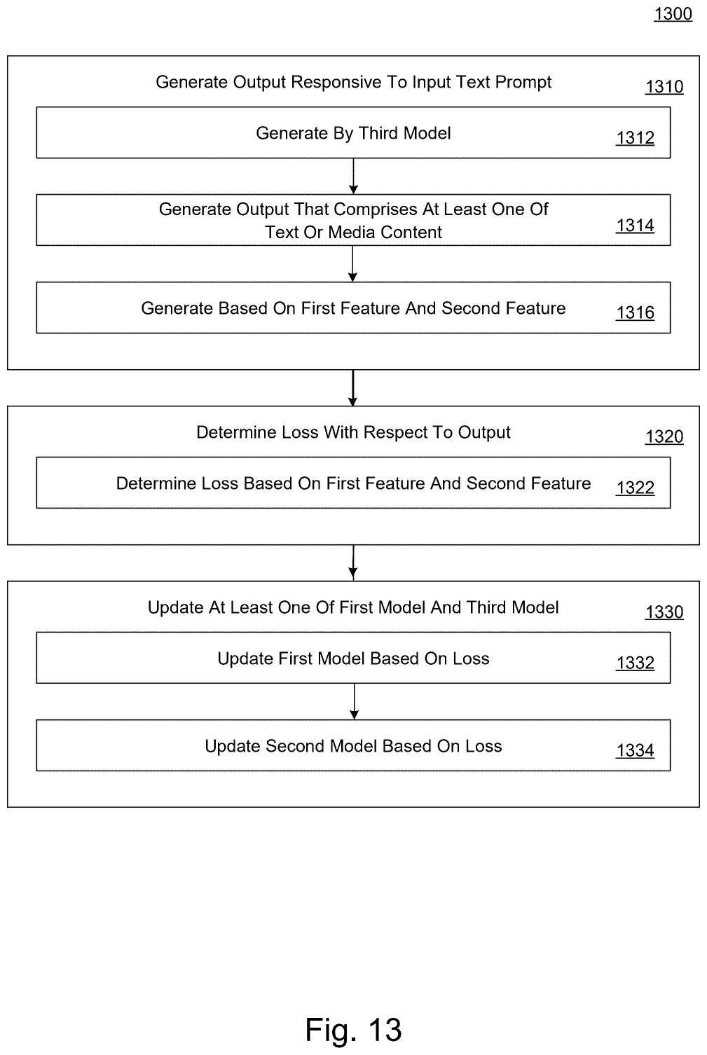

FIG. 13 depicts an example method of multi-modal query and response for medical procedures according to this disclosure. At least the system 100 can perform method 1300. At 1310, the system 100 can generate an output responsive to the input text prompt. At 1312, the system 100 can generate the output a third model. For example, the third model can correspond to the visual decoder or the text decoder 162 as discussed herein. At 1314, the system 100 can generate an output that comprises at least one of text or media content. At 1316, the system 100 can generate the output based on the first feature and the second feature. At 1320, the system 100 can determine a loss with respect to the output. At 1322, the system 100 can determine the loss based on the first feature and the second feature. At 1330, the system 100 can update at least one of the first model and the third model. At 1332, update the first model based on the loss. At 1334, the system 100 can update the second model based on the loss. For example, the system 100 can minimize the loss by minimizing at least one of a mean absolute error (MAE), root mean squared error (RMSE), mean absolute error of an inverse depth (iMAE), and root mean squared error of an inverse depth (iRMSA).

In some embodiments, the system 100 can train a machine learning model using a final loss based on a plurality of component losses. Thus, the final loss is a combination of the plurality of losses. For example, a first loss of the plurality of losses can be based on a first set of fused features provided as input to the learnable gated module 1070, and a second loss of the plurality of losses can be based on a second set of fused features provided as input to the learnable gated module 1070. For example, the learnable gated module 1070 can generate the final loss based on the characteristics of each of the first set of fused features and the second set of fused features. example, the learnable gated module 1070 can generate the final loss by combining the first loss associated with the first set of fused features and the second loss associated with the second set of fused features. The machine learning model can thus be updated using unsupervised learning (e.g., the loss functions), without labels.

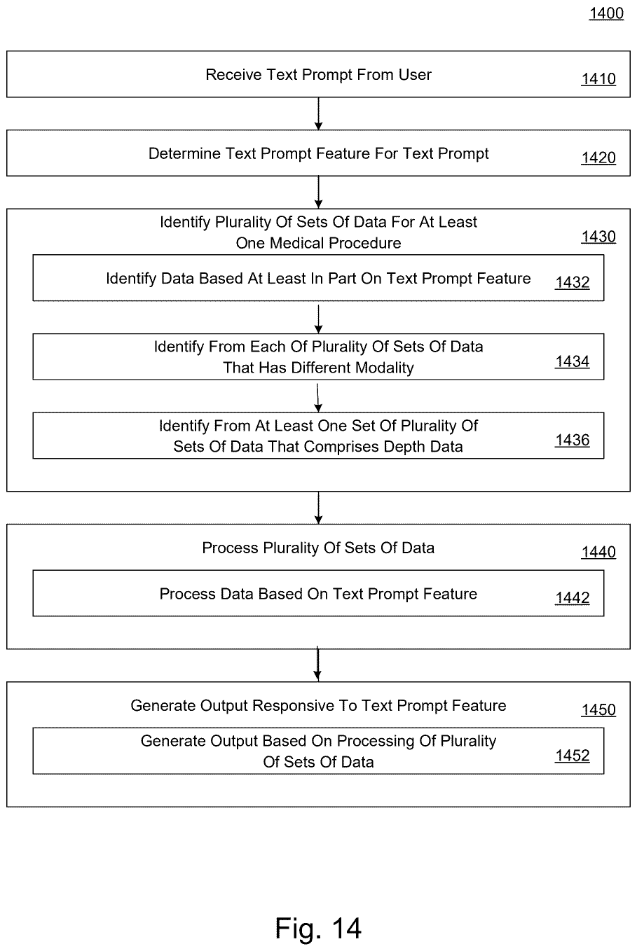

FIG. 14 depicts an example method of multi-modal query and response for medical procedures according to this disclosure. At least the system 100 can perform method 1400. At 1410, the system 100 can receive a text prompt from a user. At 1420, the system 100 can determine a text prompt feature for the text prompt. At 1430, the system 100 can identify a plurality of sets of data for at least one medical procedure. At 1432, the system 100 can identify the data based at least in part on the text prompt feature. At 1434, the system 100 can identify from each of the plurality of sets of data that has a different modality. At 1436, the system 100 can identify from at least one set of the plurality of sets of data that comprises depth data. At 1440, the system 100 can process the plurality of sets of data. At 1442, the system 100 can process the data based on the text prompt feature. At 1450, the system 100 can generate an output responsive to the text prompt feature. At 1452, the system 100 can generate the output based on the processing of the plurality of sets of data.

Having now described some illustrative implementations, the foregoing is illustrative and not limiting, having been presented by way of example. In particular, although many of the examples presented herein involve specific combinations of method acts or system elements, those acts and those elements may be combined in other was to accomplish the same objectives. Acts, elements and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items. References to “is” or “arc” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein, and are not limited to the directional indicators depicted herein. Any directional indicator depicted herein can be modified to the reverse direction, or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any clam elements.

Scope of the systems and methods described herein is thus indicated by the appended claims, rather than the foregoing description. The scope of the claims includes equivalents to the meaning and scope of the appended claims.

What is claimed is:

1. A system, comprising:
one or more processors, coupled with memory, to: receive a text prompt from a user;
generate, based at least in part on a plurality of sets of data associated with at least one medical procedure, an output corresponding to the text prompt, wherein each of the plurality of sets of data has a different modality, wherein the plurality of sets of data comprises depth data; and
provide the output for display.

2. The system of claim 1, wherein the output includes a text response and a visual annotation of video data, the text response includes a number responsive to the text prompt, and the plurality of sets of data comprises the video data.

3. The system of claim 1, wherein the output includes a text response and a portion of video data within a time period, the text response and the time period each responsive to the text prompt, and the plurality of sets of data comprises the video data.

4. The system of claim 1, wherein the output includes a text response and an image, the text response includes at least a partial description of the image and is responsive to the text prompt.

5. The system of claim 4, wherein
the partial description of the image includes a description of at least one of an absolute location or a relative location within the medical environment of a first object in the plurality of sets of data, the relative location of the first object relative to a second object in the medical environment; or
the partial description of the image includes a description of a layout of one or more objects in the plurality of sets of data, the image corresponds to a 3D reconstruction of a medical environment based on point cloud data, the medical environment corresponds to the medical procedure, and the plurality of set of data includes the point cloud data.

6. The system of claim 1, wherein the output includes a text response and a plurality of portions of video data, the text response includes a number responsive to the text prompt, a number of the plurality of portions of the video data corresponds to the number responsive to the text prompt, and the plurality of sets of data comprises the video data.

7. The system of claim 6, wherein
the number corresponds to a number of people involved in a task in a medical environment corresponding to the medical procedure, and the plurality of portions of the video data depicts one or more of the number of people involved in the task;
the number corresponds to a number of instruments used in a task in a medical environment corresponding to the medical procedure, and the plurality of portions of the video data depicts one or more of the number of instruments used in the task; or
the number corresponds to a number of events by a person involved in a task in a medical environment corresponding to the medical procedure, and the plurality of portions of the video data depicts one or more of the events.

8. The system of claim 1, wherein the output includes a text response and a data visualization output, the text response includes at least a partial description of the data visualization output.

9. The system of claim 1, the processors to:
determine a modality that is responsive to the text prompt, wherein the modality includes at least one of video data, an annotation of video data, an image, and a data visualization.

10. The system of claim 1, the processors to:
extract one or more features from one of more of the plurality of sets of data; and generate one or more fused features each including one or more of the features each having the different modality; and
generate the output based on one or more of the fused features.

11. The system of claim 1, the processors to:
select, according to a determination that the plurality of sets of data includes video data, a neural network configured to extract features from the video data; and
extract, by the neural network, the one or more features from the video data.

12. A system, comprising:
one or more processors, coupled with memory, to:
receive multi-modal data comprising video data, analytics data, and metadata for one or more medical procedures each to update one or more models;
generate, using a first model configured to detect image features, a first feature that identifies an object in the video data for a plurality of medical procedures, wherein the video includes at least one of medical staff, a patient, a robotic system or instrument, or medical environment;
generate, using a second model, a second feature that identifies features in a text prompt;
generate, by a third model and based on the first feature and the second feature, an output responsive to the input text prompt, wherein the output comprises at least one of text or media content;
determine, based on the first feature and the second feature, a loss with respect to the output; and
update at least one of the first model, the second model, and the third model based on the loss.

13. The system of claim 12, wherein the output includes a text response and a visual annotation of the video data, and the text response includes a number responsive to the text prompt.

14. The system of claim 12, wherein the output includes a text response and a portion of the video data within a time period, the text response and the time period each responsive to the text prompt.

15. The system of claim 12, wherein the output includes a text response and an image, the text response includes at least a partial description of the image and is responsive to the text prompt, and the media content comprises the image.

16. The system of claim 15, wherein the partial description of the image includes a description of a location of an object in the multi-modal data.

17. The system of claim 15, wherein the partial description of the image includes a description of a layout of one or more objects in the plurality of sets of data, and the image is based on the metadata.

18. The system of claim 12, wherein the output includes a text response and a plurality of portions of the video data, the text response includes a number responsive to the text prompt, and a number of the plurality of portions of the video data corresponds to the number responsive to the text prompt.

19. The system of claim 12, wherein the output includes a text response and a data visualization output, and the text response includes at least a partial description of the data visualization output.

20. A system, comprising:
one or more processors, coupled with memory, to: receive a text prompt from a user;
determine a text prompt feature for the text prompt;
identify, based at least in part on the text prompt feature, a plurality of sets of data associated with at least one medical procedure, wherein each of the plurality of sets of data has a different modality, wherein at least one set of the plurality of sets of data comprises depth data;
process the plurality of sets of data based on the text prompt feature; and
generate, based on the processing of the plurality of sets of data, an output responsive to the text prompt feature.
